(19) United States
(12) Patent Application Publication
(10) Pub. No.: US 2017/0312305 A1
(43) Pub. Date: Nov. 2, 2017

(54) PHARMACEUTICAL EXTRACTS AND USES THEREOF

(71) Applicant: UNIVERSITY OF SALFORD ENTERPRISES LIMITED, Salford (GB)

(72) Inventors: David Alexander PYE, Salford (GB); Olanrewaju Dorcas OGUNDIPE, Salford (GB)

(73) Assignee: UNIVERSITY OF SALFORD ENTERPRISES LIMITED, Salford (GB)

(21) Appl. No.: 15/522,700

(22) PCT Filed: Oct. 26, 2015
(86) PCT No.: PCT/GB2015/053209

§ 371 Date: Apr. 27, 2017

(30) Foreign Application Priority Data
Oct. 30, 2014 (GB) 1419354.4

(51) Int. Cl. A61K 31/727; A61K 31/726; A61K 35/618; A61K 45/06

(52) U.S. Cl. CPC A61K 31/727; A61K 45/06; A61K 31/726; A61K 35/618

(57) ABSTRACT

The invention provides an isolated extract comprising glycosaminoglycans from whelk and/or cockle species.

### TECHNICAL FIELD OF THE INVENTION

This invention relates to isolated glycosaminoglycans, particularly whelk and cockle glycosaminoglycans, their use in pharmaceutical compositions and in the treatment and prevention of cancer or cancer metastasis.

### BACKGROUND TO THE INVENTION

The extracellular matrix (ECM) was long believed to act almost solely as a support structure for cells within tissues. This view has changed markedly over the last 25 years, with matrix components shown to play critical roles in biological processes such as tissue development and remodelling, homeostasis and progression of many serious diseases. The macromolecular components of the ECM are involved in complex relationships with growth factors, cytokines and cell surface receptors that are closely associated with the cellular and molecular mechanisms involved in the growth and progression of cancer. This is mediated by the effects ECM components have on cancer cell adhesion, migration as well as the cancers invasiveness and metastatic potential. Glycosaminoglycans (GAG) containing proteins, known as proteoglycans, are major components of the ECM. Glycosaminoglycans are linear, macromolecular heteropolysaccharides, comprised of repeating disaccharide units, which are extensively modified during their synthesis. Members of the GAG family include heparan sulphate and heparin, which are glucosamine containing sulphated GAGs, chondroitin sulphate and dermatan sulphate based on a galactosamine backbone and the unsulphated GAG, hyaluronic acid.

Heparan sulphate (HS), and the closely related molecule heparin, is made up of a repeating 1,4-linked disaccharide unit. This is composed of a hexuronic acid, one of either β-D-Glucuronic acid (GlcUA) or α-L-Iduronic acid (IdoUA) and a α-D-N-acetyl (GlcNAc) or α-D-N-sulpho-glucosamine (GlcNS). In addition to N-sulphation, many of these constituent disaccharides bear one or several 0-sulphate substituents. Commonly 0-sulphation occurs at C-2 of IdoUA to form the monosaccharide α-L-Iduronic acid 2-O-sulphate (IdoUA(2S)) and/or at C-6 of GlcNS or GlcNAc to form the monosaccharides α-D-N-sulpho-glucosamine 6-O-sulphate (GlcNS(6S)) and α-D-N-acetyl-glucosamine 6-O-sulphate (GlcNAc(6S)). Sulphated disaccharides mainly occur in blocks termed ‘S-domains’, these are separated by stretches of the none sulphated disaccharide GlcUA(1-4)GlcNAc, which is predominant, and typically makes up around 50% of the total disaccharide units in HS and 80-90% in heparin.

Heparin is a heavily sulphated GAG and closely related in structure to HS. It is mainly known for its clinical use as an intravenous anticoagulant drug, which goes back to the 1940s. Since this time heparin has been absolutely vital to the progression and development of vascular and cardiac surgery, haemodialysis, organ transplantation and treatment/prevention of thromboembolism.

It is well recognised that various short chain HS oligosaccharides have the ability to interact with protein ligands and regulate their biological activity. To date, these oligosaccharides have been solely made up of, or contain clusters of, sulphated residues (S-domains). In some notable cases, e.g. antithrombin III, fibroblast growth factor-1 and fibroblast growth factor-2, the interactions/biological activity of HS oligosaccharides has been shown to be dependent on critical sequences of sulphated monosaccharides. In others cases e.g. hepatocyte growth factor/scatter factor and vascular endothelial growth factor a predominance of a particular sulphation position is required, in these cases C-6 sulphation over C-2 or N-sulphation, has been shown to be important. Many different biologically active S-domain oligosaccharides of varying length and sulphation pattern can be excised from polymeric HS through the action of the enzyme heparinase III. Oligosaccharides generated in this way contain unsaturated uronic acid residues (AUA) at their non-reducing end. Unfortunately, the number of oligosaccharides produced from natural HS sources is truly vast (billions of possible structures), as a result of variation in sulphate group number and position. S-groups are primarily present in 3 positions on each disaccharide (N—S, 6-S, 2-S). Therefore, a naturally occurring decasaccharide could have over 200 million possible structures.

GAGs have been used in the preparation of pharmaceutical compositions for the treatment of various diseases, including use as anti-cancer agents, but the vast number of possible GAG structures mean that it is not possible to predict which specific GAG structures will be effective against any particular condition or disease, and as GAGs can be isolated from many hundreds of different organisms this problem is compounded. The search for any particular GAG effective against any specific disease cannot be easily narrowed down on the basis of the organism of origin or particular structure of GAG, even if a similar GAG is known to have the desired properties.

WO2012/045750 discloses the use of a combination of a mucant and GAG to treat proliferative conditions including cancer;

US2002/016308A discloses the use of heparin-like GAGs to prevent and treat thrombosis associated with vascular injury, the GAGs being derived from mast-cells;

CN1321469A discloses the use of glycoproteins and GAGs from pearl oysters and their anti-tumour effect; and

KR20110132746A discloses the isolation of GAGs from sea slugs and their potential anti-cancer properties.

While GAGs have therefore been proposed for use as pharmaceutical agents against a wide variety of diseases, determining and isolating an effective GAG against any particular disease or ailment is not possible from a simple review of know GAGs and their origin.

It would therefore be advantageous to be able to isolate further GAGs from origins not yet investigated, and to utilise suitable GAGs as therapeutic agents in the prevention and treatment of diseases including cancer and cancer metastasis.

It would also be advantageous to identify new origins for GAGs effective in the prevention or treatment of cancer and cancer metastasis.

It is therefore an aim of embodiments of the invention to overcome or mitigate at least one problem of the prior art.

### SUMMARY OF THE INVENTION

According to a first aspect of the invention there is provided an isolated extract comprising glycosaminoglycans from whelk and/or cockle species.

According to a second aspect of the invention there is provided use of the isolated extract of the first aspect of the invention in a pharmaceutical composition for the prevention and/or treatment of cancers and/or cancer metastasis.

According to a third aspect of the invention there is provided a pharmaceutical composition comprising glycosaminoglycans from whelk and/or cockle species.

According to a fourth aspect of the invention there is provided use of a pharmaceutical composition of the third aspect of the invention for the prevention and/or treatment of cancer and/or cancer metastasis.

Glycosaminoglycans are a type of polysaccharide commonly referred to as GAGs and shall be used interchangeably hereinafter.

The isolated GAG extract may comprise a mixture of GAGs, which may include heparin and/or heparan sulphate (or heparan sulphate-like GAG). In some embodiments the isolated GAG extract comprises heparin and/or heparan sulphate or heparan sulphate-like GAG. In some embodiments the extract comprises at least one heparan sulphate (“HS”) and at least one non-heparin GAG, such as chondroitin sulphates (“CS”) and/or dermatan sulphates (“DS”). In some embodiments the extract comprises HS and CS, HS and DS or HS, DS and CS. In other embodiments the extract may comprise an HS-like sulphated GAG, and may comprise a HS-like sulphated GAG and at least one of a CS or DS. The HS-like sulphated GAG may comprise at least a disulphide bridge. In some embodiments the GAGs (which in some embodiments comprise HS, DS, CS or any mixture thereof) comprises at least one lower molecular weight component comprising a molecular weight in the range of around 0.25 KDa to around 35 KDa, such as between around 0.5 KDa and around 25 KDa or around 0.5 KDa to around 10 KDa. In other embodiments the lower molecular weight component may have a molecular weight in the range 0.25 KDa to 300 KDa, 0.5 KDa to 300 KDa, 1 KDa to 300 KDa, 5 KDa to 300 KDa, 10 KDa to 300 KDa, 25 KDa to 300 KDa, 100 KDa to 300 KDa or 150 KDa to 250 KDa. In some embodiments the GAGs (which may comprise HS, CS, DS or any mixture thereof) comprises at least one higher molecular weight component comprising a molecular weight in the range of between around 300 KDa and around 1200 KDa such as between around 500 KDa and around 1000 KDa, or around 750 KDa to around 1200 KDa. In some embodiments the GAGs, (which may comprise HS, CS, DS or any mixture thereof) comprises a mixture of the lower molecular weight component and the higher molecular weight component, and may comprise a component having a molecular weight of around 0.25 KDa to 300 KDa and a component having a molecular weight of around 500 KDa to 1200 KDa (or 750 KDa to 1200 KDa). In some embodiments the GAGs may have a first component having a molecular weight of around 0.25 KDa to 300 KDa, a second component having a molecular weight of around 300 KDa to 750 KDa and a third component having a molecular weight in the range 1000 KDa to 1200 KDa. The isolated GAG extract may consist essentially of heparan sulphates which may be as described above, and may consist essentially of the lower molecular weight and/or higher molecular weight components.

The GAGs (which may comprise HS, CS, DS or any mixtures thereof) may comprise a pharmaceutically acceptable salt thereof, such as an alkali metal salt, for example a sodium or potassium salt, an earth alkaline metal salt, for example a calcium or magnesium salt, an ammonium salt or a salt of an organic base. The GAGs (which may comprise HS, CS, DS or any mixtures thereof) may also comprise a solvated form, such as a hydrated form for example, which possesses the desired anti-cancer and/or anti-metastatic activity.

The pharmaceutical compositions of the invention may comprise a pharmaceutically acceptable carrier, diluent, excipient or any combination thereof.

The pharmaceutical compositions of the invention may be in a form suitable for oral use, which may be a tablet, lozenge, hard or soft capsule, suspension, emulsion, dispersible or non-dispersible powder or granules, syrups, elixir, colloidal suspension, or any combination thereof.

The pharmaceutical composition may be in the form suitable for topical application, such as a cream, gel, ointment, salve, suspension or any combination thereof, for example.

The pharmaceutical composition may be in the form suitable for administration parenterally, such as an intravenous, subcutaneous, intramuscular or intraperitoneal injection, for example.

The pharmaceutical composition may be in the form suitable for inhalation, insuffation, or a suppository.

In some embodiments the pharmaceutical composition is in a form suitable for injection.

Cancers and cancer cells suitable for treatment include breast cancer (including oestrogen receptor negative cell-lines such as MDANQ01, MDA MB-231 and MDA-468); leukaemia (such as leukaemia cell lines including MOLT-4 and K562); cervical cancer; ovarian cancer; and colon cancer. Other cancers may also be suitable.

Without being bound by any particular theory it is believed that the isolated GAGs from whelks and cockles of the present invention exert their therapeutic effect, at least in part, by inducing apoptosis in various cancer cell-lines. Therefore, in a further aspect of the present invention there is provided an isolated extract comprising glycosaminoglycans from whelk and/or cockle species for use in the induction of apoptosis of cancerous cells.

In a further aspect, the present invention provides a method of inducing a apoptosis in cancerous cells, the method comprising administering an effective amount of an isolated extract comprising glycosaminoglycans from whelk and/or cockle species, or a pharmaceutically acceptable salt thereof.

In some embodiments of the present invention, the isolated extract of glycosaminoglycans and pharmaceutical compositions comprising glycosaminoglycan extracts are those derived from whelk species, in particular for use in the prevention and/or treatment of cancers and/or cancer metastasis, such as breast cancer, leukaemia and cervical cancer.

The pharmaceutical compositions of the invention may be obtained by conventional procedures using conventional pharmaceutical excipients common well known in the art. Thus, pharmaceutical compositions intended for use in the parenteral administration of glycosaminoglycans from whelk and/or cockle species may contain, for example, a suitable vehicle and, optionally one or more preservative agent.

It will be appreciated that a suitable vehicle used for the GAGs derived from whelk and/or cockle species should be one that is well tolerated by the subject to whom it is given, and enables delivery of the GAGs to the desired site of action.

It will be appreciated that the amount of GAGs required is determined by biological activity and bioavailability which in turn depends on the mode of administration, the physiochemical properties of the GAG extract and whether the GAG is being used as a monotherapy or in a combined therapy. The frequency of administration will also be influenced by the above mentioned factors and particularly the half-life of the GAGs within the subject being treated. Optimal dosages of the GAGs to be administrated may be determined by those skilled in the art, and will vary with the particular extract being used, the species from which it is derived, the strength of the preparation, the mode of administration and the advancement of the disease conditions.

In one embodiment, the amount of GAGs in a pharmaceutical composition of the invention is in an amount from about 0.01 mg to about 800 mg. In another embodiment, the amount of the GAGs is in an amount from about 0.1 mg to about 80 mg.

Generally, a daily dose of between 0.01 μg/kg of bodyweight and 1.0 g/kg of bodyweight of the GAGs maybe used for the treatment of cancer and/or cancer metastasis. Daily doses may be given as a single administration (e.g. a single daily injection or infusion) or may require administration two or more times during any specific day.

The GAGs of the invention any pharmaceutical compositions comprising the same may be applied as a sole therapy or may involve, in addition, conventional, surgery, radiotherapy or chemotherapy. Such chemotherapy may include one or more or the following categories of anti-cancer agents:
1. Other anti-proliferative/antineoplastic drugs and combinations thereof (for example cis-platin);
2. Cytostatic agents (for example tamoxifen);
3. Anti-invasion agents;
4. Inhibitors of growth factor function;
5. Angiogenic agents (for example those which inhibit the effects of vascular endothelial growth factor);
6. Antisense therapies;
7. Gene therapies;
8. Immuno therapy approaches;
9. One or more GAGs having anti-cancer properties isolated from species other than whelk and/or cockle.

The term “combination” is used to refer to simultaneous, separate or sequential administration of two or more therapies and/or agents.

The whelk GAGs may be extracted from whelks of the family Buccinidae such as from the genus Buccinum (“Busycon Whelks”). The whelk may be Buccinum undatum. In other embodiments the GAGs may be extracted from whelks of the family Muricidae (such as Nucella lapillus and Ocenebra erinacea), Nassariidae (such as Nassarius (Hinia) reticulate and Nassarius incrassatus), or Littorinidae (such as Littorina littorea).

The cockle GAGs may be extracted from cockles of the family Cardiidae and may be from one or more of the following genus; Acanthocardia, Acrosterigma, Americardia, Cardium, Cerastoderma, Clinocardium, Corculum, Ctenocardia, Dinocardium, Discors, Fragum, Fulvia, Laevicardium, Lophocardiium, Lunulicardia, Lyrocardium, Microcardium, Nemocardium, Papyridea, Parvicardium, Plagiocardum, Pratulum, Ringicardium and Serripes.

The cockle may be Cerastoderma edule.

According to another aspect of the invention there is provided an isolated heparan sulphate or heparan sulphate-like GAG extract from whelk and/or cockle species. The heparan sulphate or heparan sulphate-like GAG may comprise a lower molecular weight component having a molecular weight in the range of around 0.25 KDa to around 300 KDa, 0.5 KDa to around 300 KDa, around 1 KDa to around 300 KDa, around 5 KDa to around 300 KDa, around 10 KDa to around 300 KDa, around 0.25 KDa to around 100 KDa around 0.25 KDa to around 50 KDa, 0.25 KDa to around 35 KDa, such as between around 0.5 KDa and around 25 KDa or around 0.5 KDa. The heparan sulphate or heparan sulphate-like GAG may comprise a higher molecular weight component comprising a molecular weight in the range of between around 300 KDa and around 1200 KDa such as between around 500 KDa and around 1000 KDa or around 750 KDa to around 1200 KDa. In some embodiments the heparan sulphate or heparan sulphate-like GAG comprises a mixture of the lower molecular weight component and the higher molecular weight component or may comprise a component having a molecular weight of around 0.25 KDa to 300 KDa and a component having a molecular weight of around 500 KDa to 1200 KDa (or 750 KDa to 1200 KDa). In some embodiments the heparin sulphate or heparan sulphate-like GAG may have a first component having a molecular weight of around 0.25 KDa to 300 KDa, a second component having a molecular weight of around 300 KDa to 750 KDa and a third component having a molecular weight in the range 1000 KDa to 1200 KDa. The isolated GAG extract may consist essentially of heparan sulphates or heparan sulphate-like GAG which may be as described above, and may consist essentially of the lower molecular weight and/or higher molecular weight components.

According to another aspect of the present invention there is provided a pharmaceutical composition comprising an isolated heparan sulphate or heparan sulphate-like GAG extract from whelk and/or cockle species.

According to another aspect of the invention there is provided a pharmaceutical composition comprising whelk and/or cockle heparan sulphate or heparan sulphate-like GAG.

According to yet another aspect of the invention there is provided use of a pharmaceutical composition comprising heparan sulphate or heparan sulphate-like GAG from whelk and/or cockle species for the prevention and/or treatment of cancer and/or cancer metastasis.

According to another aspect of the invention there is provided the use of glycosaminoglycans for the prevention and/or treatment of leukaemia.

According to a further aspect of the invention there is provided a pharmaceutical composition comprising glycosaminoglycans for the prevention and/or treatment of leukaemia.

The glycosaminoglycans may comprise heparin and/or heparan sulphate, heparan sulphate and CS, heparan sulphate and DS, or heparan sulphate DS and CS. In some embodiments the glycosaminoglycans comprise heparan sulphate. The glycosaminoglycans may be as described herein above for any of the other aspect of the invention, and may be derived from cockle and/or whelk species as described and defined hereinabove.

According to a further aspect of the invention there is provided an isolated extract comprising sulphated polysaccharides from whelk and/or cockle species.

According to a further aspect of the invention there is provided the use of an isolated extract comprising sulphated polysaccharides from whelk and/or cockle species in the prevention and/or treatment of cancer and/or cancer metastasis.

According to a further aspect of the invention there is provided a pharmaceutical composition comprising sulphated polysaccharides from whelk and/or cockle species. The pharmaceutical composition may be used in the prevention and/or treatment of cancer and/or cancer metastasis.

The isolated extracts and pharmaceutical compositions may be as described and defined above.

### DETAILED DESCRIPTION OF THE INVENTION

In order that the invention may be more clearly understood, embodiments thereof will now be described, by way of example only, with reference to the accompanying drawings, of which:

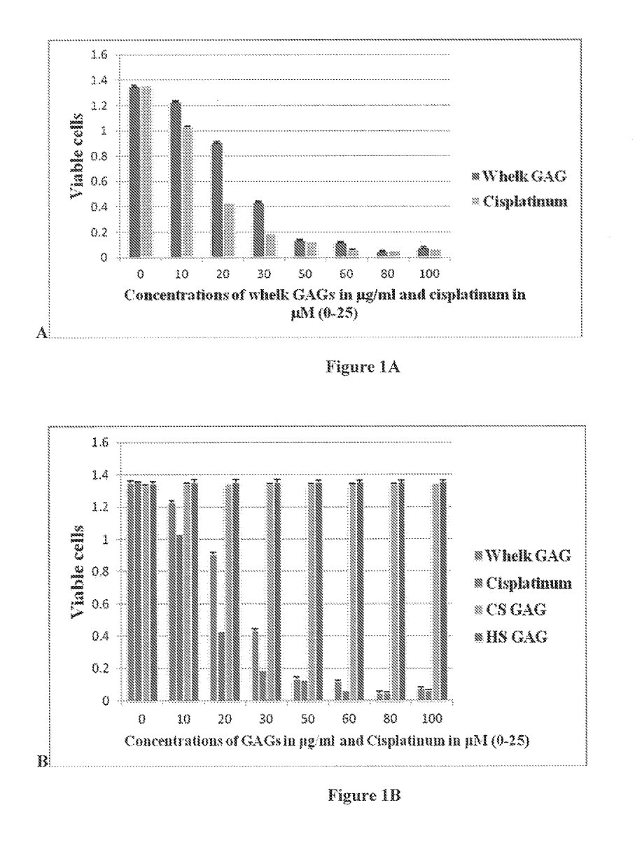

FIG. 1A is an MTT cell viability assay for MDANQ01 cells after treatment with whelk GAGs and cisplatin;

FIG. 1B illustrates MTT cell viability assay results for MDANQ01 cells after treatment with commercial porcine GAGs and cisplatin;

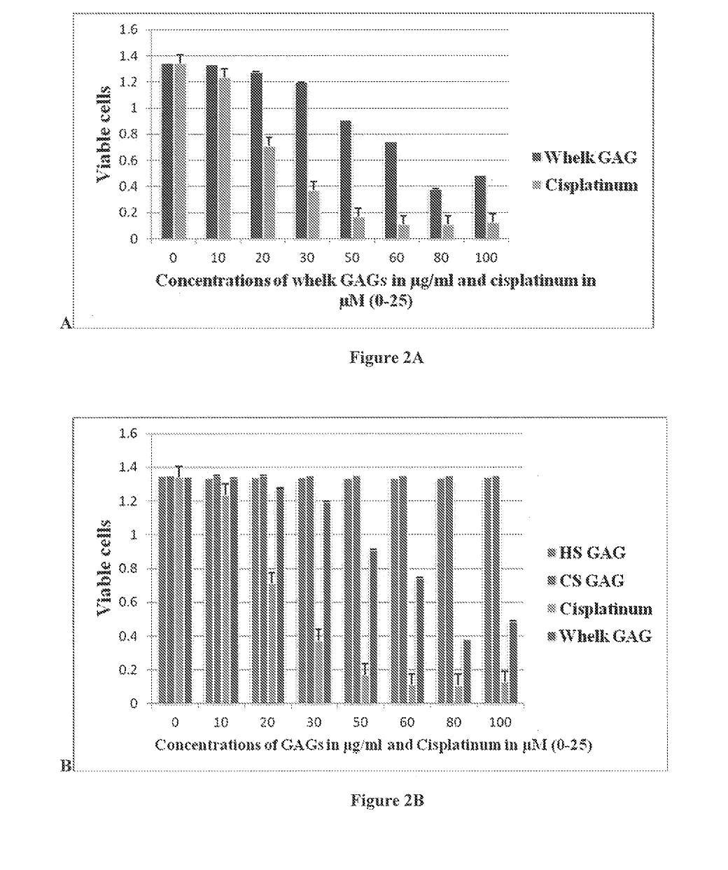

FIG. 2A illustrates MTT cell viability assay for MDA-468 cells after treatment with whelk GAGs and cisplatin;

FIG. 2 B illustrates the results of an MTT cell viability assay for MDA-468 cells after treatment with commercial (porcine) GAGs, whelk GAGs and cisplatin;

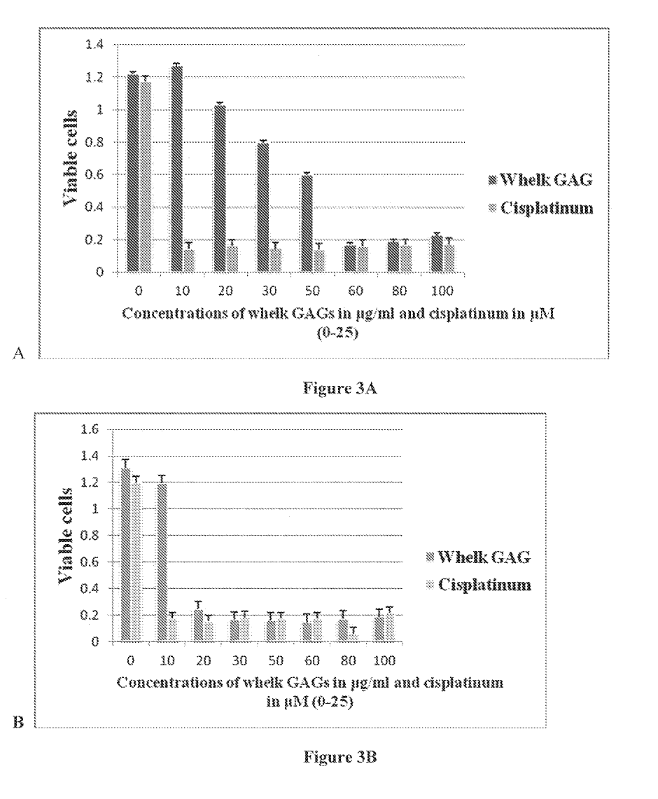

FIG. 3A illustrates the results of an MTT cell viability assay for MOLT-4 leukemia cells after treatment with whelk GAGs and cisplatin;

FIG. 3B illustrates the results of an MTT cell viability assay for K562 leukemia cells after treatment with whelk GAGs and cisplatin;

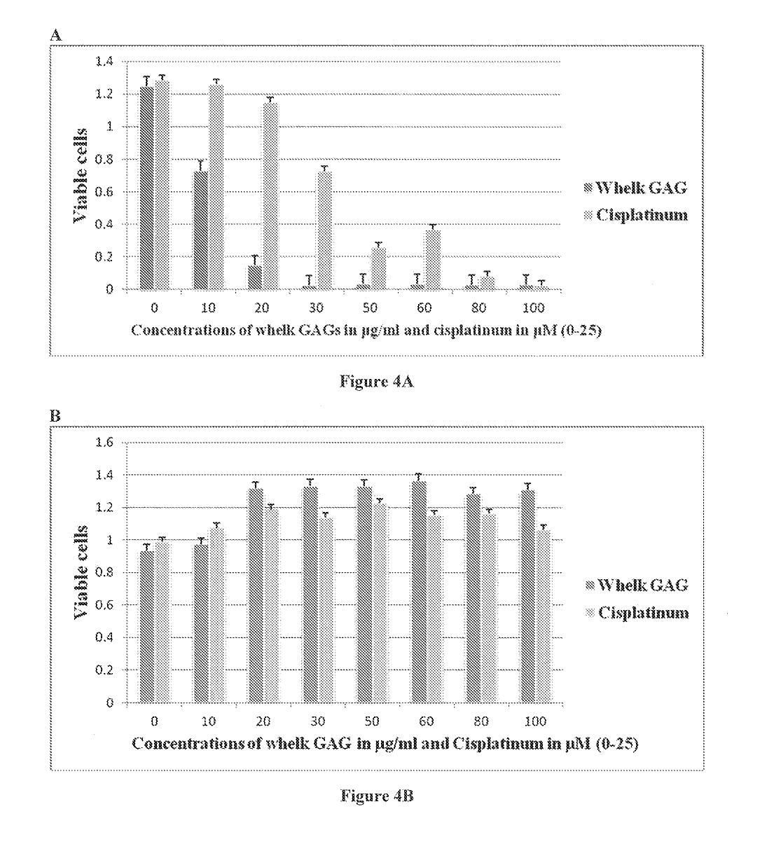

FIG. 4A illustrates an MTT cell viability assay of HeLa cells after treatment with whelk GAGs and cisplatin;

FIG. 4B illustrates an MTT cell viability assay of normal fibroblast 3T3 cells after treatment with whelk GAGs and cisplatin;

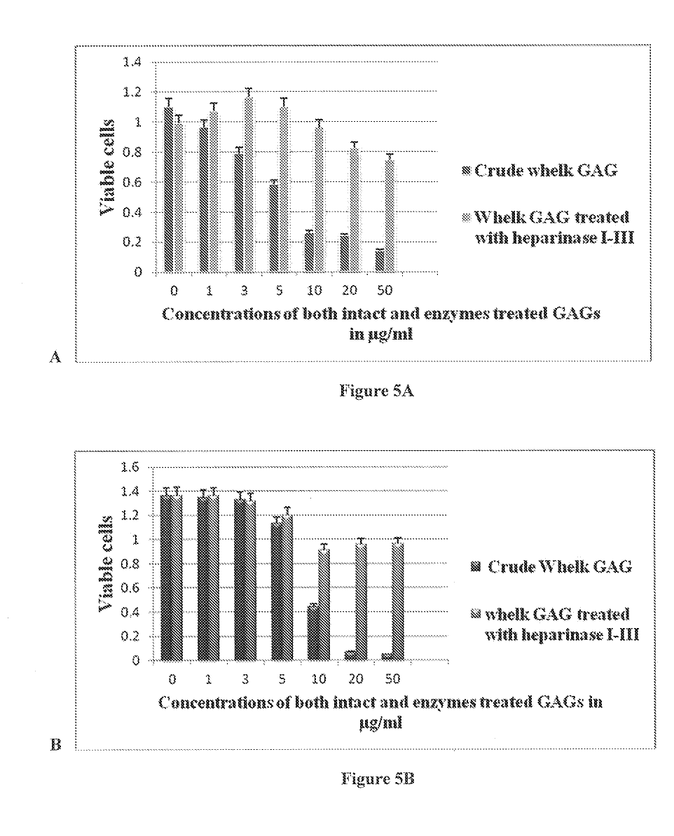

FIG. 5A illustrates the results of an MTT cell viability assay for MDA468 breast cancer cells after treatment with enzyme depolymerised whelk GAGs and crude whelk GAGs;

FIG. 5B illustrates the results of an MTT cell viability assay for MDANQ01 breast cancer cells after treatment with enzyme depolymerised whelk GAGs and crude whelk GAGs;

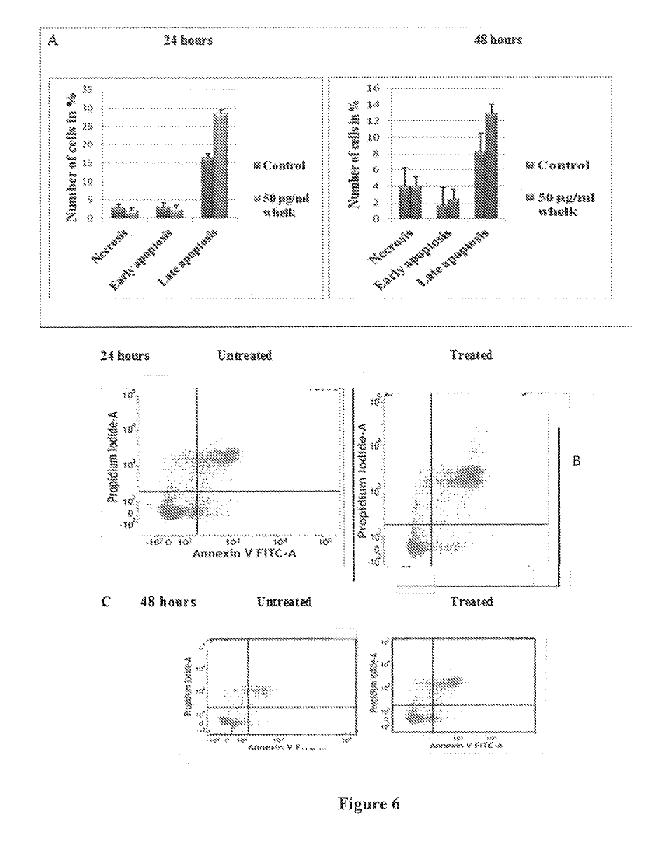

FIGS. 6A-6C illustrate the results of the detection of apoptotic MDA468 breast cancer cells treated with crude whelk GAGs by Annexin V Staining;

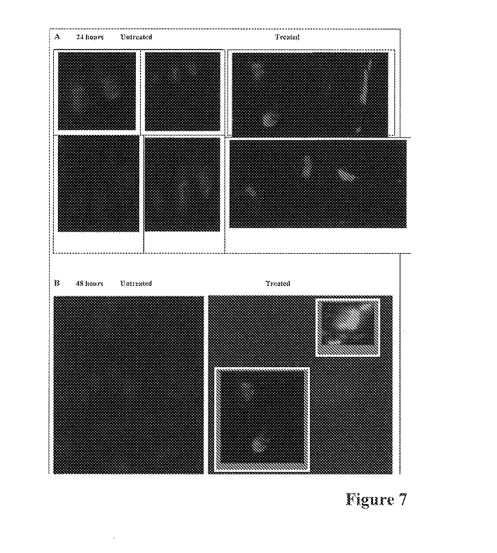

FIGS. 7A-7B illustrate the results of detection of apoptotic MDA468 cells treated with crude whelk GAG mixtures by DAPI fluorescent microscopy;

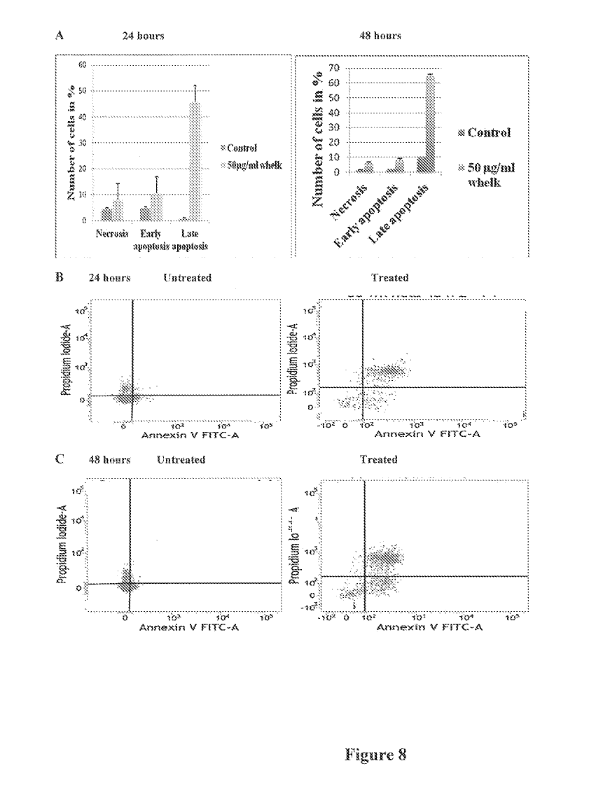

FIG. 8 illustrates the detection of apoptotic HeLa cervical cancer cells treated with crude whelk GAG mixtures by Annexin V Staining;

FIGS. 9A-9B illustrate the detection of apoptotic HeLa cells treated with crude whelk GAG mixtures by DAPI fluorescent microscopy;

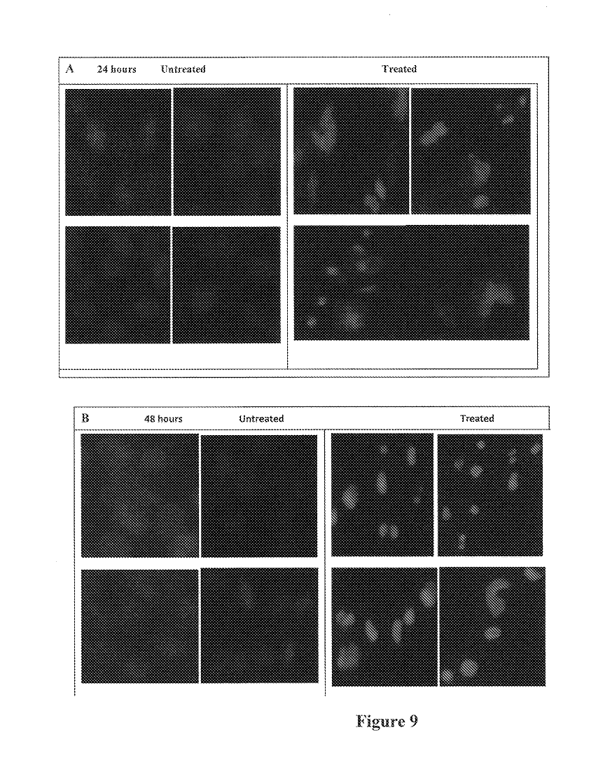

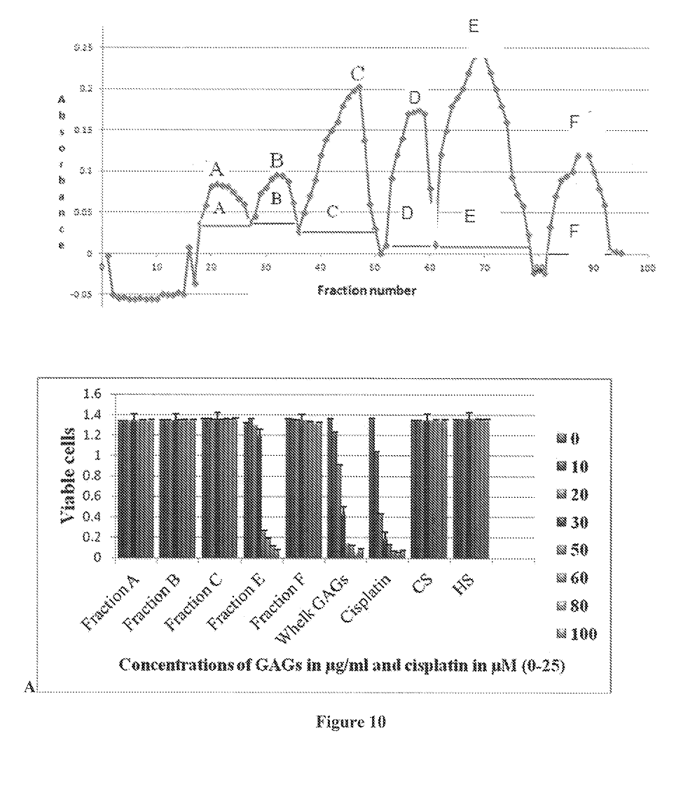

FIG. 10 illustrates the results of ion-exchange chromatography separation of whelk GAG mixtures;

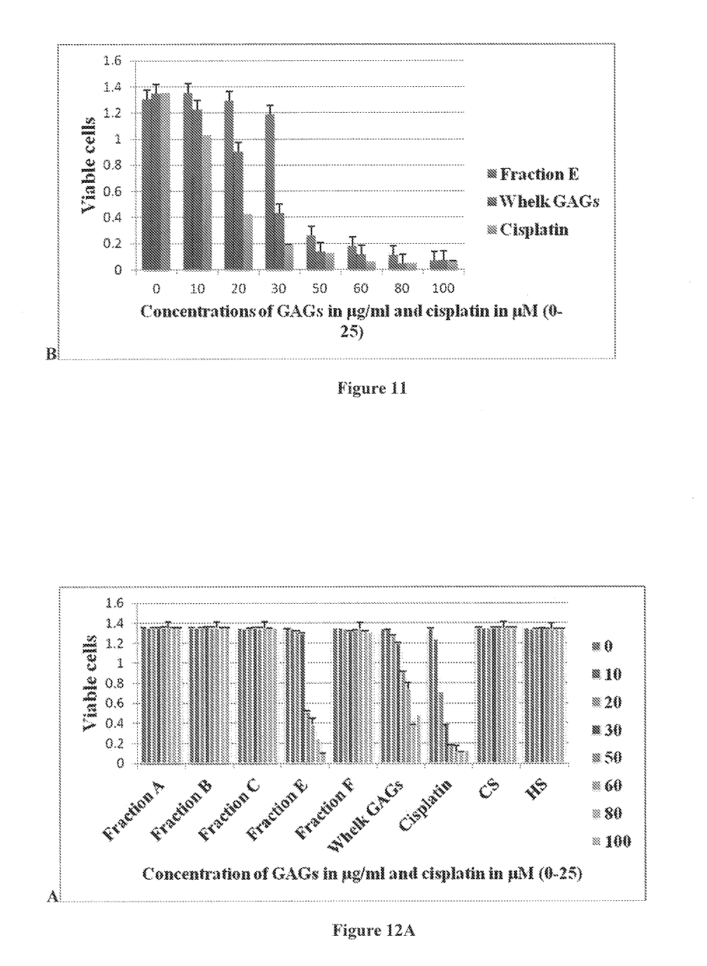

FIG. 11A-11B illustrate the results of an MTT cell viability assay for MDANQ01 breast cancer cells after treatment with GAGs. FIG. 11A illustrates the results after treatment with crude extracts of GAGs, while FIG. 11B illustrates the results using fraction E of the crude whelk GAGs, crude whelk GAGs per se and cisplatin;

FIG. 12A-12B illustrate the results of concentration-dependent effect of purified whelk “fraction E” GAGs on MDA468 breast cancer cell viability. FIG. 12A illustrates cells treated with commercial GAGs, whelk purified fractions and cisplatin, while FIG. 12B illustrates cells treated with “fraction E”, crude whelk GAGs and cisplatin;

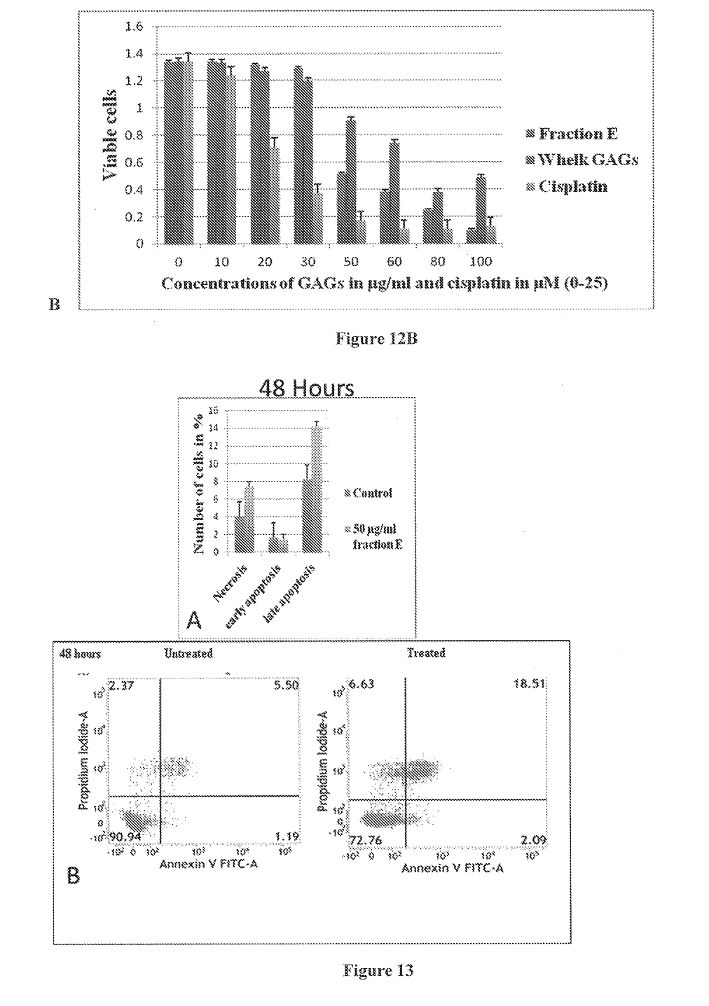

FIG. 13 illustrates the results of detection of time dependent apoptotic MDA468 breast cancer cells treated with purified whelk “fraction E” by Annexin V Staining;

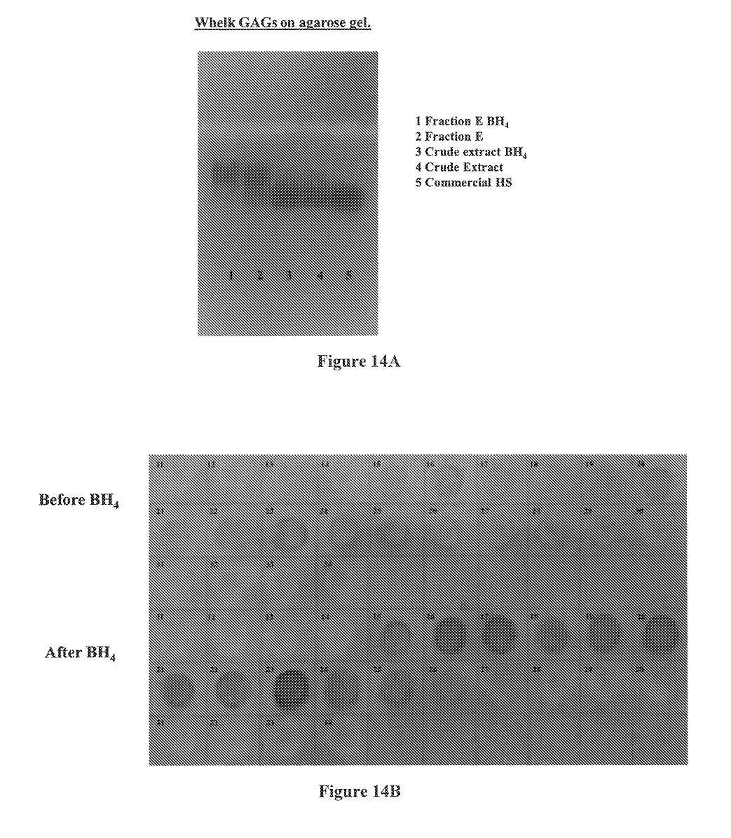

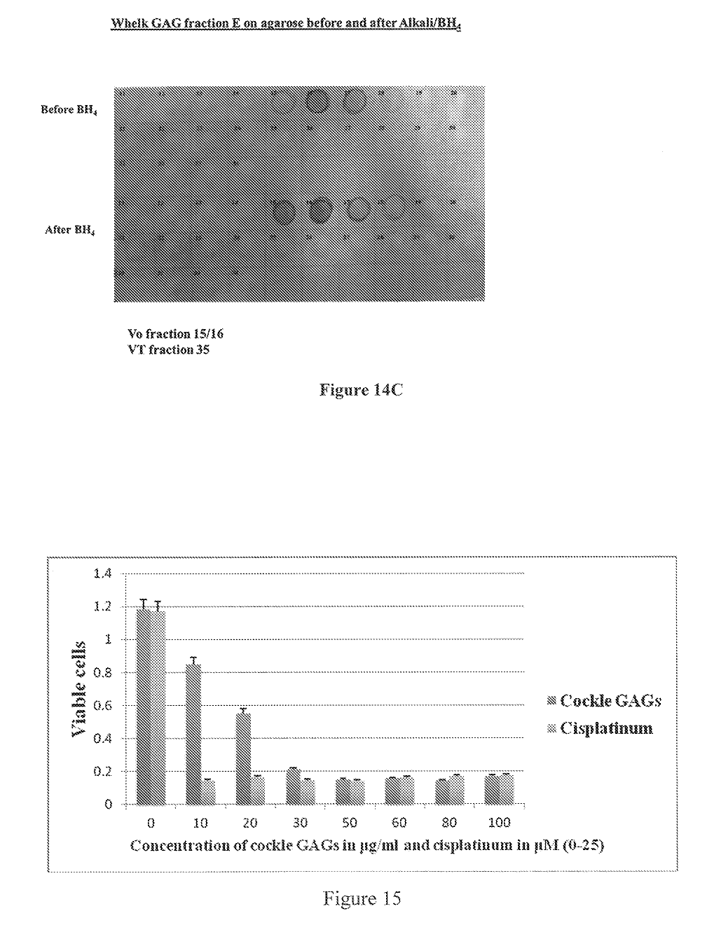

FIG. 14A is an agarose gel showing the separation of GAGs and purified whelk “fraction E” by agarose electrophoresis;

FIG. 14B is a cellulose acetate blot of crude whelk GAGs from Superose-12 column gel-filtration before and after alkali/BH₄ treatment;

FIG. 14C is a cellulose acetate blot which shows the results of Superose-12 column gel-filtration of whelk GAG “fraction E” before and after alkali/BH₄ treatment.

FIG. 15 is a bar chart illustrating the concentration dependent effects of cockle GAGs on MOLT-4 cell viability;

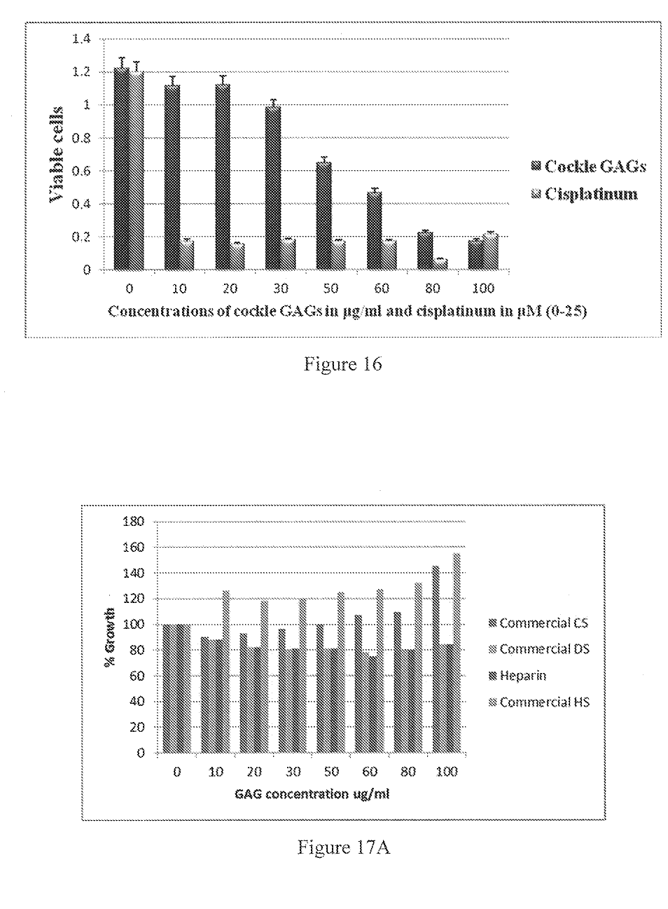

FIG. 16 is a bar chart illustrating the concentration dependent effects of cockle GAGs on K562 cell viability;

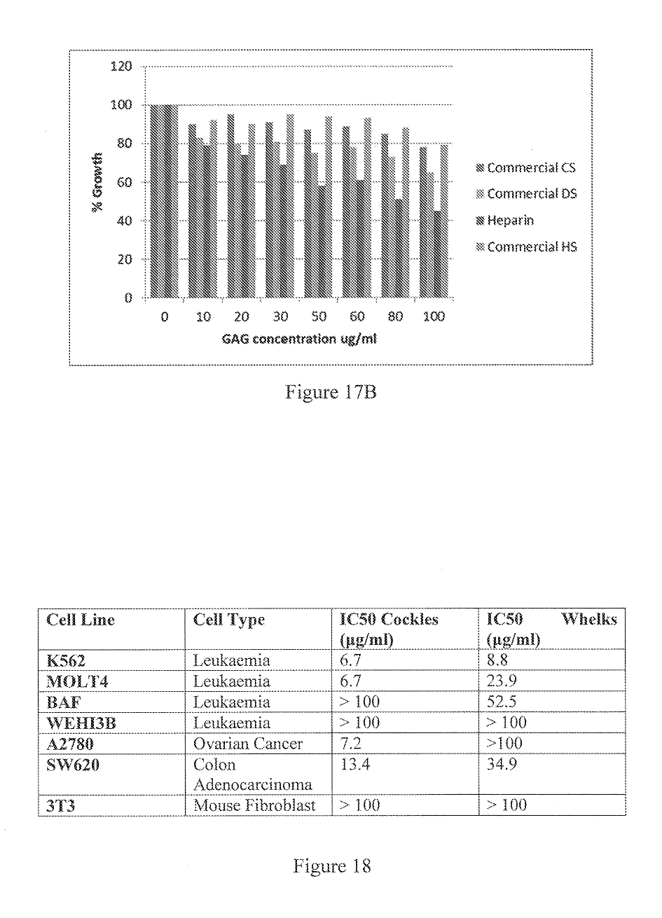

FIG. 17A-17B are bar charts illustrating the concentration-dependent effects of commercial GAGs on K562 cell viability and on MOLT-4 cell viability;

FIG. 18 illustrates a table showing the IC₅₀ values for cockle and crude whelk GAG extracts on various cancer cell lines, taken from different batches of GAG extracts.

### EXAMPLE 1

An example of the isolation of whelk GAG extracts according to the invention and their use in a pharmaceutical composition for the treatment of cancers will now be described.

### Materials and Methods

### Preparation of GAG Extracts

Whelk samples (Buccinum undatum) from the Irish Sea were sourced from local fish markets in Fleetwood, UK and were defatted using three 24 h extractions with acetone and dried. The fat-free dried cockles and whelks were crushed into a fine powder using an industrial blender. Approximately 4 g of dried, defatted, pulverised powder was suspended in 40 ml of 0.05 M sodium carbonate buffer (pH 9.2) and 2 ml of the proteolytic enzyme Alcalase (Type XIV, ≧3.5 units/mg; 10 mg/ml) was added to the suspension in order to detach the carbohydrate chains from proteins. The suspension was shaken for 48 h at 200 rpm at 60° C. The digestion mixture was cooled to 4° C., and trichloroacetic acid added to a final concentration of 5% (to remove non degraded proteins/peptides and nucleic acids). The sample was mixed, allowed to stand for 10 min, and then centrifuged for 20 min at 8000 rpm. The supernatant was recovered by decanting and the precipitate discarded Three volumes of 5% potassium acetate in ethanol were added to one volume of supernatant. After mixing, the suspension was stored overnight at 4° C. and then centrifuged for 30 min at 8,000 rpm. The supernatant was discarded, and the precipitate was washed with absolute alcohol. The precipitate was dissolved in 40 ml of 0.2 M NaCl, centrifuged for 30 min at 8,000 rpm and the insoluble material discarded. GAGs were recovered from the supernatant by addition of 0.5 ml of a 5% solution of cetylpyridinium chloride and the precipitate collected by centrifugation.

The precipitate was dissolved in 10 ml of 2.5 M NaCl followed by the addition of 5 volumes of ethanol and the precipitate removed by centrifugation for 30 min at 10,000 rpm. The crude GAG precipitate was then dissolved in distilled water and dialysed as described below.

Crude GAGs obtained from the extraction procedure were desalted using dialysis tubing pre-treated with 100 mM EDTA. The dialysis tubes containing GAG extracts were dialysed against 100 volumes of distilled water for 72 h under constant agitation with the distilled water changed at 24 h intervals. After 72 hours, the desalted GAG extracts were freeze dried and stored at −20° C. for further use.

### Purification of Crude GAGs by Anion Exchange Chromatography

GAG samples (10 mg) derived from the shellfish samples were dissolved in 10 ml distilled water and manually injected onto a column (1.5×5 cm) of DEAE-sephacel equilibrated with 50 mM sodium phosphate buffer, pH 7.0. The column was eluted using a linear gradient of 0-3 M NaCl in 50 mM phosphate buffer, pH 7.0 over a hundred minutes at flow rate of 1 ml/min. The elution was monitored spectrophotometrically at 210, 232 and 254 nm and lml fractions collected. Peak were pooled, freeze dried and finally desalted using PD 10 column.

### Enzyme Digestion of GAGs

Samples of GAGs (1 mg/ml) were treated with either heparinases or chondroitinase ABC lyase (30 mIU) and incubated for 24 hours at 37° C.

### Agarose Electrophoresis

Agarose gels (0.5% in 50 mM1,2-diaminopropane/acetate pH9.0) were prepared just prior to electrophoresis Samples corresponding to 10 μg GAG were prepared in volumes of 10 μl and applied to the gels. Electrophoresis was performed in 50 mM 1,2-diaminopropane at a constant voltage of 100V for 40 minutes. Following separation, gels were stained in 0.5% Azure A in water for 10 minutes, and de-stained in water to visualise bands.

### Superose-12 Size Exclusion Chromatography

Samples (at 10 mg/ml) in a total volume of 20 μl of water were applied to a Sepharose-12 column, eluted in 0.2M ammonium hydrogencarbonate at a flow rate of 0.5 ml/minute and 0.5 ml fractions collected. Column Vo and Vt values were established by application of bromophenol blue and sodium dichromate (1 mg/ml).

Vo fraction 15/16

Vt fraction 34/35

Fractions were assessed for GAG content by cellulose acetate dot blotting.

### Cellulose Acetate Dot Blotting of GAGs

Cellulose acetate sheets were marked with a grid of 1 cm² squares using a pencil. Multiple applications of each fraction were applied to the membrane using a pipette. The acetate sheet was dried thoroughly between applications in an oven at 75° C. for 5 minutes each. Following the final drying the sheet was stained using a solution of 0.5% Azure A stain for 10-20 seconds, then de-stained in tap water until a good contrast between stained dots and background was achieved.

### Beta-Elimination of GAG Chains Using Alkaline/Borohydride.

Samples in water (buffer-free) were combined 1:1 with a solution containing 200 mM NaOH/2M NaBH₄, and incubated at 37° C. overnight. Borohydride was neutralised by drop-wise addition of acetic acid until gas release ceased. Samples were then de-salted on PD-10 columns in water, and dried by centrifugal evaporation before re-solubilising in water.

### Determination of Cells Viability (MTT Assay).

The breast cancer cell MDA 468 and MDANQ01, K562 and human lymphoblastic cell line (MOLT-4) were grown in RPMI 1640 medium supplemented with 1% L-glutamine, 1% of 100 Units/ml penicillin and 0.1 mg/ml streptomycin and 10% inactivated FCS. While the ovarian cell line HeLa and fibroblast cell line 3T3 were grown in DMEM medium supplemented with 20% inactivated FCS, 1% L-glutamine, 1% of 100 units/ml penicillin & 0.1 mg/ml streptomycin. All cell lines were maintained in a humidified incubator with an atmosphere of 95% air and 5% CO₂ at 37° C.

Cell viability was determined by the MTT (3-(4,5 dimethylthiazol2-yl)-2,5 diphenyltetrazolium bromide) test method. Cells were cultured overnight in 96-well plates (2.0×10⁴ cells/well) containing 100 μl medium prior to treatment with crude GAG extract, commercial GAGs and cisplatinum at 37° C. This was followed by addition of 100 μl of fresh medium containing various concentrations of GAGs (0-100 μg/ml) or cisplatinum (0-25 mM) into each well, and incubated for another 96 hrs. The metabolic activity of each well was determined by the (MTT) assay and compared to those of untreated cells. At the end of the incubation period, 50μl of MTT solution (5 mg/ml in PBS) was added to each well and incubated for three hours at 37° C. After incubation the supernatants were carefully aspirated, then 200 μl of DMSO was added to each well and the plates agitated to dissolve the crystal product. Cell viability was determined based on mitochondrial conversion of 3[4,5-dimethylthiazol-2-yl] 2,5-diphenyltetrazolium bromide (MTT) to formazan. The amount of MTT converted to formazan is indicative of the number of viable cells. The plates were gently agitated until the colour reaction was uniform and the absorbance was measured at 570 nm using a multi-well plate reader, Sigma Plot 2000 software was used for data analysis. The cell viability effects from exposure of cells to each concentration of crude whelk GAGs, commercial GAGs and cisplatinum were analysed as percentages of the control cell absorbance, which were obtained from control wells plated in RPMI, 1% L-glutamate and 10% FCS media. The average cell survival obtained from triplicate determinations at each concentration was plotted as a dose response curve. The 50% inhibition concentration (IC₅₀) of the active substances was determined as the lowest concentration which reduced cell growth by 50% in treated compared to untreated cells.

### Flow Cytometry Analyses of DNA Content (Cell Cycle Analysis).

Cells were seeded into 25-cm plastic sterile culture flasks, and incubated for 24 hours prior to treatment with different concentrations of crude and purified GAGs samples. Cells were harvested at different time intervals (8-48 hours), centrifuge at 1500 rpm for 5 minutes, washed three times with phosphate buffer saline (PBS) and fixed in ice cold 70% (v/v) ethanol for 30 minutes. Before analysis, cells were centrifuged to remove ethanol and washed with PBS three times. Cells were then incubated at room temperature with 50 μl of Ribonuclease A (RNase) (50 mg/ml) and finally stained with a solution containing 50 g/ml propidium iodide (PI) for 30 min in the dark. The stained samples were analysed with Partec flow cytometer. The cell cycle distribution was evaluated on DNA plots by Partec cyflow space version 2.4 software

### Annexin V FITC Apoptosis Detection

Cells were seeded into sterile 6 well plates at 5×10⁵ cells/ml, and incubated with or without GAG extracts at 37° C. Cells were harvested at different time intervals (8-48 hours), centrifuged at 1500 rpm for 5 minutes, washed twice with cold PBS and resuspended in 1× binding buffer (0.1M HEPES, 1.4 M NaCl and 25 mM CaCl₂) at a concentration of 1×10⁶ cells/ml. Cells (100 μl) were transferred into 5 ml culture tubes and stained with Annexin V FITC (5 μl) and PI (10 μl). The stained cells were gently vortexed then incubated in the dark for 15 minutes at room temperature. 400 μl of 1× binding buffer was added to each tube prior to analysis using a BD flow cytometer.

### DAPI Fluorescent Microscopy for Apoptosis Detection

Cells were seeded into sterile 8 well plates at 5×10⁵ cells/ml, and incubated with or without GAG samples at 37° C. Cells were harvested at different time intervals (8-48 hours). The harvested cells were washed twice with cold PBS followed by 1× binding buffer (0.1M HEPES, 1.4 M NaCl and 25 mM CaCl₂). The cells were immediately stained with DAPI and incubated in the dark for 10 minutes before finally being rinsed with binding buffer.

### Results

### Anti-Cancer Activity Data of Whelk GAGs on Breast Cancer Cell Lines

FIG. 1 shows the results of a MTT cell viability assay for MDANQ01 cells after treatment with shellfish polysaccharides (GAG). Cells were incubated with various concentrations of GAGs for 96 hours after which cell viability was measured using MTT assay (A) whelk crude GAGs and cisplatin as a positive inhibitory control, (B) commercial GAGs and cisplatin as a positive control. Each value is presented as the mean SEM of three independent determinations. The bars in each chart are presented as relative values in comparison to untreated cells.

FIG. 2 shows the result of an MTT cell viability assay for MDA-468 cells after treatment with shellfish polysaccharides (GAG). Cells were incubated with various concentrations of GAGs for 96 hours after which cell viability was measured using MTT assay (A) whelk crude GAGs and cisplatin as a positive inhibitory control, (B) commercial and crude whelk GAGs with cisplatin as a positive control. Each value is presented as the mean SEM of three independent determinations. The bars in each chart are presented as relative values in comparison to untreated cells.

The results indicated the inhibitory activities of crude whelk GAG extracts on breast cancer cell lines MDA NQ01 and MDA-468 in vitro (FIGS. 1 and 2). Typical IC₅₀ values for whelk GAGs on MDANQ01 and MDA-468 breast cancer cell lines were around 25 μg/ml and 70 μg/ml respectively. The results also showed that the MDANQ01 cell growth is markedly inhibited by whelk GAGs at concentrations as low as 10-20 μg/ml with almost 100% growth inhibition at 100 μg/ml as shown in FIG. 1A. The MDA-468 cell line showed a diminished response to the crude whelk GAG, indicating some selectivity in cell response. The observed inhibitory effect of whelk GAGs on the cell lines compared favourably with cisplatinum (a common anti-cancer agent) at a concentration range 1-25 μM with IC₅₀'s of 0.63 μM (MDA NQ01) and 0.88 (MDA-468). In order to examine further this unusual GAG related activity, commercial (porcine) samples of GAG (HS and CS) (FIGS. 1B and 2B) were tested for their anti-cancer effects and it can clearly be seen that they have none. A search of the literature has shown no cytotoxic effects associated with typical GAG chains.

These results suggest that the whelk derived GAG's possesses a unique and as yet undiscovered structure/activity relationship with regard to inhibition of the growth of breast cancer cells and shows great potential in the treatment of this disease.

### Anti-Cancer Activity of Whelk GAGs on Leukaemia Cell Lines

FIGS. 3A and 3B shows the results of the MTT cell viability assay for MOLT-4 and K562 leukemia cells respectively after treatment with GAGs. (A) MOLT-4 leukemia cells treated with crude whelk GAGs and cisplatin as a positive inhibitory control, (B) K562 leukemia cell line treated with crude whelk GAGs and cisplatin as a positive control.

Crude whelk GAG extracts were tested on two leukaemia cell lines. The data obtained from these results show that the crude whelk GAG extracts have a strong growth inhibitory effects on leukemia cells lines MOLT-4 and K562 (FIGS. 3A and 3B). The growth inhibitory activities of the crude whelk on K562 erythroleukaemia cell line, typical IC₅₀ values are around 15 μg/ml. concentrations in the range of 20-100 μg/ml produced very strong inhibition and static growth as seen in FIG. 3B. The crude whelk GAGs also inhibited the growth of MOLT-4 leukaemia cells in a concentration dependent manner with a much higher value, IC₅₀ values, typically around 55 μg/ml (FIG. 3A). The anti-cancer activities of the crude extract with the two leukaemia cell lines again compare favorably with cisplatinum. Once again the commercial (porcine) GAGs showed no anticancer activity with either of the two leukaemia cell lines. More, importantly, the different activities displayed by the crude whelk extract on these two cell lines also supported the selectivity previously seen with the two breast cancer cell lines. This selectivity may well be important in targeting the effects of these molecules towards different tumour cells or indeed normal tissue.

### Effect of Crude Whelk GAG Mixtures on Growth of Normal Fibroblast Cells (3T3) In Vitro

FIGS. 4A and 4B show the results of MTT cell viability assay on normal mouse fibroblast 3T3 cells after treatment with GAGs. Cells were incubated with various concentrations of GAGs for 96 hours after which cell viability was measured using MTT assay (FIG. 4A) HeLa cells treated with crude whelk GAGs and cisplatin as a positive inhibitory control, (FIG. 4B) 3T3 normal fibroblast cells treated with crude whelk GAGs and cisplatin as a positive control. Each value is presented as the mean SEM of three independent determinations. The bars in each chart are presented as relative values in comparison to untreated cells.

The 3T3 cells were exposed to a range of different concentrations of the crude whelk GAGs for times ranging up to 96 hours. Crude whelk GAGs did not show any growth inhibitory effect on 3T3 cell proliferation after this time (FIG. 4B), in contrast to those activities observed for cancer cells. Moreover, the effects of crude whelk GAGs on 3T3 cells compare favourably with the results obtained for commercial GAGs. These results suggest that the whelk derived GAG's possesses a unique and as yet undiscovered activity against the growth of cancer cells.

### Effect of Heparinase I, II, & III Enzymes on Anti-Cancer Activity of Whelk GAG Mixtures on MDA468 and MDANQ01 Breast Cancer Cell Lines.

FIGS. 5A and 5B show the results of MTT cell viability assay for MDANQ01 and MDA468 breast cancer cells respectively after treatment with enzyme depolymerised whelk GAG samples.

Cells were incubated with various concentrations of GAGs for 96 hours after which cell viability was measured using the MTT assay. (FIG. 5A) shows the result of MDA468 cells treated with whelk heparinase I-III enzyme digest (oligosaccharides) and cisplatin as a positive inhibitory control, (FIG. 5B) shows the results of MDANQ01 cells treated with whelk heparinase I-III enzyme digest (oligosaccharides) and cisplatin as a positive inhibitory control. Each value is presented as the mean SEM of three independent determinations. The bars in each chart are presented as relative values in comparison to untreated cells.

The crude whelk GAG was depolymerised with heparinase I, II & III enzymes as this should reduce sensitive polysaccharide chains, such as HS, into disaccharides along with a minor amount of short resistant tetrasaccharides. These small HS fragments have previously been shown to be devoid of biological activities seen with intact HS chains and are useful in identifying the class of GAG involved in selected biological activities of complex polysaccharides. The heparinase treated whelk GAG samples were incubated with the two breast cancer cell lines (MDANQ01 and MDA 468) for 96 hours as described in the materials and methods section. The results obtained show anti-cancer effects of whelk GAGs on both cell lines in contrast to what was obtained for the intact crude extract (FIG. 5).

### Crude Whelk GAG Mixtures Induce Apoptosis in MDA468 Breast Cancer Cells

FIGS. 6A to 6c show the results of detection of Apoptotic MDA468 breast cancer cells treated with crude whelk GAG mixtures by Annexin V Staining.

MDA468 breast cancer cells were treated with crude whelk GAGs for 24 and 48 hours and then stained using Annexin V-FITC and propidium iodide provided in the Annexin V-FITC Apoptosis Detection Kit (see materials and methods). The combination of Annexin V-FITC and propidium iodide allows for the distinction between early apoptotic cells (Annexin V-FITC positive), late apoptotic and/or necrotic cells (Annexin V-FITC and propidium iodide positive), necrotic (PI stained positive) and viable cells (unstained). (FIG. 6A) shows the number of cells which had undergone apoptosis when treated with or without crude whelk GAGs for 24 and 48 hours respectively. (FIG. 6B) shows MDA468 cell treated with or without crude whelk GAGs for 24 hours. (FIG. 6C) shows MDA468 cell treated with or without crude whelk GAGs for 48 hours.

The results show that whelk GAG induced cell death in MDA468 breast cancer cells but the mechanism of cell death induced by this crude GAGs polysaccharide is not yet known. An apoptotic detection kit was used to study the mechanism of cell death following treatment with crude whelk GAG extract, using annexin V FITC conjugated antibodies and flow cytomety. Cells were incubated, with varying concentrations of the crude GAG, and cells analysed at different time intervals to determine the presence and number of apoptotic cells. The results show that the crude GAG extract increased the proportion of breast cancer cells undergoing apoptosis (late apoptosis) as a proportion of the rate of cell death caused by the crude extract on the cells (FIG. 6A). Treatment with 50 μg/ml of whelk GAGs increased the percentage of cells stained positive for both annexin V FITC and PI from 16.48 (control) to 28.40 (50 μg/ml) after 24 hours. 48 hours incubations with the crude whelk GAGs also slightly increased the number of cells stained with annexin V FITC from 1.64% (control) to 2.4% (50 μg/ml) and the number of cells stained positive for both annexin V FITC and PI was also marginally increased from 8.2% (control) to 12.89% (50 μg/ml). The apoptotic inducing effect on both the treated and untreated cells was confirmed by staining with DAPI and viewing under the fluorescent microscope.

### DAPI Staining and Fluorescent Microscopy of MDA-468 Cells

FIGS. 7A and 7B show the results of detection of apoptotic MDA468 cells treated with crude whelk GAG mixtures by DAPI fluorescent microscopy.

MDA468 cells treated with crude whelk GAGs were stained with DAPI and morphology of apoptotic cell nuclei was observed using a fluorescence microscope. Images were photographed at the same exposure time under a ×40 objective with Hamatsu 1394 ORCA-285 camera. (FIG. 7A) MDA468 cells treated with or without crude whelk for 24 hours. (FIG. 7B) MDA468 cells treated with or without crude whelk GAGs for 48 hours.

To further understand whether the anticancer effects seen with the whelk GAG extract was due to an induction of apoptosis, we employed standard DAPI staining methodology followed by fluorescent microscopy. MDA-468 cells were grown with or without 50 μg/ml of crude whelk GAGs for 24 and 48 hours and the cells subsequently analysed by DAPI staining. The cells incubated in ordinary FCS media without crude whelk GAGs showed no signs of apoptosis or oncosis and exhibited brightly fluorescing round, blue-stained nuclei (FIG. 7A). In contrast, after 24 and 48 hours treatment with crude whelk GAGs, there were a numbers of cells with condensed and fragmented nuclei, an indication of apoptosis as indicated in FIGS. 7A and 7B.

Trypsinisation which is known to cause membrane damage is one of the major steps involved in annexin V FITC apoptosis detection assay and could potentially be responsible for the false anexin V FITC positive stain. However, the above result obtained from the DAPI staining confirms that the crude GAG mixtures are inducing apoptosis in cancer cell lines.

### Effect of Crude Whelk GAG Mixtures on Apoptosis in HeLa Cells

FIGS. 8A to 8C show the results of detection of Apoptotic HeLa cervical cancer cells treated with crude whelk GAG mixtures by Annexin V Staining.

HeLa cells were treated with crude whelk GAGs for 24 and 48 hours and then stained using Annexin V-FITC and propidium iodide provided in the Annexin V-FITC Apoptosis Detection Kit. The combination of Annexin V-FITC and propidium iodide allows for the distinction between early apoptotic cells (Annexin V-FITC positive), late apoptotic and/or necrotic cells (Annexin V-FITC and propidium iodide positive), necrotic (PI stained positive) and viable cells (unstained). FIG. 8A shows the percentage of HeLa cells in apoptosis when treated with or without crude whelk GAGs for 24 and 48 hours respectively. (FIG. 8B) HeLa cell treated with or without crude whelk GAGs for 24 hours. (FIG. 8C) HeLa cells treated with or without crude whelk GAGs for 48 hours.

The results show that crude whelk GAG extracts also caused cell death in the HeLa cell line, this cell line has previously been shown to be resistant to entering apoptosis. It was therefore chosen to investigate the ability of the whelk GAG extract to circumvent the apoptotic block in these cells that is partly responsible for their immortality.

Anexin V staining and flow cytometry was used as previously described for the MDA 468 cells. Interestingly the apoptotic effects of the crude whelk GAG extract was more pronounced and better established with this cell line. Following 24 hours incubation with the crude whelk extracts almost 50% of the HeLa cells were seen to be apoptosis (late apoptosis) in this with an additional 10% in early (FIG. 8).

48 hours incubations with the crude whelk GAG induced almost 70% late apoptosis and almost 10% early (FIG. 8). DAPI staining and fluorescent microscopy was performed to confirm this observation.

### DAPI Staining and Fluorescent Microscopy of MDA-468 Cells

FIG. 9 shows the detection of apoptotic HeLa cells treated with crude whelk GAG mixtures by DAPI fluorescent microscopy.

HeLa cells treated with crude whelk GAGs were stained with DAPI and the morphology of apoptotic cell nuclei was observed using a fluorescence microscope. Images were photographed at the same exposure time under a ×40 objective with Hamatsu 1394 ORCA-285 camera. (A) HeLa cells treated with or without crude whelk for 24 hours. (B) HeLa cells treated with or without crude whelk GAGs for 48 hours.

DAPI staining was performed to confirm the apoptotic effects seen on HeLa cells using the annexin V marker. HeLa cells were treated with and without 50 μg/ml of crude whelk GAGs for 24 and 48 hours before staining with DAPI. The cells incubated in ordinary FCS media without crude whelk GAGs showed no signs of apoptosis and exhibited brightly fluorescing round, blue-stained nuclei (FIG. 9). In contrast cells treated with crude whelk GAG showed significant apoptotic traits. Treated cells contained condensed and fragmented nuclei, a typical indication of apoptosis (FIG. 8). The combined results of apoptosis detections by annexin V FITC and DAPI stains suggest that crude whelk GAGs induces cell death in HeLa cell via apoptosis.

### Anticancer Activities of Whelk GAG Fractions Obtained by Ion-Exchange Chromatography

FIG. 10 shows the results of Ion-exchange chromatography separation of Whelk GAG mixtures.

Samples were applied to a DEAE-Sephadex ion-exchange column and resolved by a linear 0-3 M NaCl gradient in 50 mM sodium phosphate buffer pH 7.0 at a flow rate of 1 ml/min for 100 min. Fractions were collected and the absorbance monitored at wavelengths of 254 nm. Fractions were pooled as indicated, desalted and lyophilized before further analysis.

Further investigation of the activities of the crude whelk GAG by ion-exchange separation of the crude GAG mixture was investigated in an attempt to purify the active component by Ion-exchange chromatography. Ion-exchange chromatography is routinely used to separate GAG mixtures into their respective GAG family members e.g. HS, CS, and DS. Six major peaks were identified and designated as fractions A-F (FIG. 10). These fractions were shown, by specific enzymatic digestion, to contain the typical members of the GAG family (Table 1). When tested in the MTT cell viability assay against both breast cancer cell lines, only a single fraction (E) showed any anti-cancer activity (FIGS. 11 and 12).

Table 1 shows the tabulated results of Ion-exchange chromatographic separation of whelk GAG mixtures.

| | | | | | Nacl |
|---|---|---|---|---|---|
| Fraction | Fraction | | Percentage | Elution time | gradient |
| name | number | GAGs type | yield (%) | (minutes) | (M) |
| A | 27-37 | HS | 19.90 | 27-37 | 0.9-1.24 |
| B | 39-47 | CS/DS | 13.50 | 39-47 | 1.26-1.62 |
| C | 49-62 | CS/DS | 18.00 | 49-62 | 1.63-2.1 |
| D | 65-73 | Unknown | 16.00 | 65-73 | 2.14-2.5 |
| E | 75-88 | HS | 23.30 | 75-88 | 2.56-2.9 |
| F | 89-94 | Unknown | 9.30 | 89-94 | 2.91-3.0 |

FIGS. 11A and 11B show the results of MTT cell viability assay for MDANQ01 breast cancer cells after treatment with GAGs.

Cells were incubated with various concentrations of GAGs for 96 hours after which cell viability was measured using MTT assay (A) MDANQ01 breast cancer cell line treated with crude whelk, commercial GAGs, whelk's purified fractions and cisplatin as a positive control. (B) MDANQ01 breast cancer cells treated with fraction E, crude whelk GAGs and Cisplatin as a positive inhibitory control. Each value is presented as the mean SEM of three independent determinations. The bars in each chart are presented as relative values in comparison to untreated cells.

FIGS. 12A and 12B shows the concentration-dependent effect of purified whelk fraction E GAGs on MDA468 breast cancer cell viability.

Cells were incubated with various concentrations of cockle GAGs for 96 hours after which cell viability was measured using MTT assay (A) cells treated with commercial GAGs, whelk's purified fractions and cisplatin as a positive control. (B) cells treated with fraction E, crude whelk GAGs and Cisplatin as a positive inhibitory control. Each value is presented as the mean SEM of three independent determinations. The bars in each chart are presented as relative values in comparison to untreated cells.

### Apoptotic Effects of Fraction E on MDA-468 Cells

FIG. 13 shows the results of detection of time dependent apoptotic MDA468 breast cancer cells treated with purified whelk fraction E by Annexin V Staining.

MDA468 cells were treated with purified whelk fraction E for 48 hours and then stained using Annexin V-FITC and propidium iodide provided in the Annexin V-FITC Apoptosis Detection Kit

Anexin V staining and flow cytometry was used as previously described for the MDA 468 cells and the crude whelk GAG mixtures. It can be clearly seen that the ability to promote cell death through apoptosis lies with Fraction E.

### Whelk GAG Structural Analysis

FIG. 14A shows the sseparation of GAGs and purified whelk fraction E by agarose electrophoresis. 50 mM 1,2-diaminopropane/acetate pH9.0, 100V for 60 mins, 20 ug per well, Azure A stained.

Gel-filtration using a Superose-12 HR10/30 was used to investigate the chain length of material contained in both the crude whelk GAG extract and the purified fraction E. Calibration of the Superose-12 column with comparable GAG molecular weight standards is impossible, as these don't exist. The only alternative is to use globular proteins as standards. These proteins are tight compact structures, unlike the linear GAG chains, which occupy a much larger volume relative to their molecular weight than that seen with proteins. This will, therefore, always lead to an overestimate of the molecular weight of GAG chains, as determined from their elution volume. In fact a commercial heparin sample of average molecular weight 15 kDa had a calculated molecular weight of 25 kDa when eluted from this column.

Fractions from the Superose-12 column where blotted onto a cellulose acetate sheet and stained with azure A in order to identify GAG containing fractions. The crude whelk extract showed three main peaks when eluted from the Superose-12 column (FIG. 14B). Their calculated molecular weights, determined from their elution volume were: Peak 1, range 1200-1000 kDa, Peak 2, range 750-300 kDa and Peak 3, range 200-100 kDa. The smallest GAG like chains eluted from the column had a molecular weight of approximately 30 kDa.

Fraction E was also eluted from the Superpose-12 column (FIG. 14C) and two main peaks were seen: a major component, Peak 1, range 1200-750 kDa and a minor species, Peak 2, range 300-100 kDa. The results mirrored those seen with the agarose gel, in which an intensely stained high molecular weight species is seen along with a much fainter faster moving band (FIG. 14C).

The use of the pre-packed Superose 12 column allows direct comparison of GAG chains from batch to batch isolations. Also more accurate retrospective molecular weight calculations, based on Superose-12 elution volumes, may be possible in the future should commercial GAG samples with predetermined molecular weights become available.

FIG. 14C shows the effect of Beta-elimination on the crude whelk GAG extract. The major components were recoverable after alkaline borohydride treatment. These showed no marked difference in elution pattern on gels compared to starting material, indicative of a starting material with only very small peptides attached in the first instance. The presence of high (greater than 750-1200 kDa) and low molecular weight (approximately (100-300 kDa) species can clearly be seen in the active fraction E (lanes 1 and 2). Comparison of the crude GAG extract (lanes 3 and 4) Fraction E (lanes 1 and 2) indicated that the high molecular weight component found in fraction E is a minor component of the original crude extract.

The forgoing example illustrates isolation of crude GAG extracts from whelk species, and a purification of a higher molecular weight component thereof (above 250 KDA), and their use in the treatment of a wide range of cancers including breast cancer cell-lines, leukaemia and cervical cancer. In addition, it can be clearly seen from the results in FIG. 14C that a lower molecular weight fraction also includes significant anti-cancer properties.

### EXAMPLE 2

Isolation of cockle GAG extracts according to the invention and their use in pharmaceutical compositions for the treatment of cancers was undertaken. The materials and methods were as described for Example 1, save for the GAG extracts being isolated from cockle species (Cerastoderma edule, from the Irish Sea, and sourced from local fish markets in Fleetwood, UK) rather than whelk species.

### Results

The anticancer effects of crude cockle GAGs on an acute lymphoblastic leukaemia cell line (MOLT-4) was determined after exposure to increasing concentrations of crude cockle GAGs (10-100 μg/ml) for 96 hours using the MTT assay. Crude cockle GAGs significantly decreased cell viability of MOLT-4 cells with a typical IC₅₀ values of 15 μg/ml (FIG. 15).

The anticancer effects of crude cockle GAGs on a human chronic myelogenous leukemia cell line (K562) was determined after exposure to different concentrations of crude cockle GAGs (10-100 μg/ml) for 96 hours using the MTT assay. Crude cockle GAGs had a lesser effect on this cell line with typical IC₅₀ values of approximately 50 μg/ml (FIG. 16).

### EXAMPLE 3

Commercial GAGs (obtained from porcine mucosa) were also tested for anticancer activity on two leukemia cell lines (MOLT-4 and K562). The data shown in FIGS. 17A and 17B (against K562 and MOLT-4 respectively) clearly shows a variety of effects from weak anticancer activity, to significant cellular growth promotion. This is in stark contrast to known data with two breast cancer cell lines MDA468 and MDANQ01, which showed no inhibitory or stimulatory activity when treated with the same commercial GAGs. The anticancer effects of the commercial GAGs is weak in comparison to that seen for the cockle GAG extracts but nevertheless there is an effect. IC₅₀ values for the commercial GAGs were not reached with the concentration ranges (0-100 μg/ml) used previously with the cockle GAG extracts, with the exception of heparin that showed an IC₅₀ value of around 90 μg/ml for the MOLT-4 cell line only.

### EXAMPLE 4

Whelk and cockle GAGs isolated as per Examples 1 and 2 where tested for anticancer activity against a range of cancers. FIG. 18 shows a summary of calculated IC₅₀ values from a number of separate batches of cockle and GAG extracts. The results show repeatable potent anticancer activities across a number of different cell lines other than breast cancer and leukaemia with significant selectivity observed with both the cell lines and the origin of the GAG extract.

The above embodiments are described by way of example only. Many variations are possible without departing from the scope of the invention as defined in appended claims.

1. An isolated extract comprising glycosaminoglycans from whelk and/or cockle species.

2. An isolated extract as claimed in claim 1 comprising at least one heparan sulphate or heparan sulphate-like GAG.

3. An isolated extract as claimed in claim 1 comprising one or more further glycosaminoglycans other than heparan sulphate or heparan sulphate-like GAG.

4. An isolated extract as claimed in claim 1 wherein the glycosaminoglycans comprise at least one component comprising a molecular weight in the range of 0.25 KDa to 300 KDa, preferably 0.25 KDa to 35 KDa.

5. An isolated extract as claimed in claim 1 wherein the glycosaminoglycans comprise at least one component comprising a molecular weight in the range of between 300 KDa and 1200 KDa.

6. An isolated extract as claimed in claim 4 wherein the glycosaminoglycans comprise a mixture of a 0.25 KDa to 300 KDa component and a 300 KDa to 1200 KDa component.

7. An isolated extract as claimed in claim 2 comprising heparan sulphate, and at least one of chondroitin sulphate and dermatan sulphate.

8. An isolated extract as claimed in claim 1 consisting essentially of heparan sulphate.

9. Use of an isolated extract of claim 1 in a pharmaceutical composition for the prevention and/or treatment of cancers and/or cancer metastasis.

10. A pharmaceutical composition comprising glycosaminoglycans from whelk and/or cockle species.

11. A pharmaceutical composition as claimed in claim 10 wherein the glycosaminoglycans comprise isolated extracts from whelk and/or cockle species.

12. A pharmaceutical composition as claimed in claim 10 comprising a pharmaceutically acceptable carrier, diluent, excipient or any combination thereof.

13. A pharmaceutical composition as claimed in claim 10 in a form suitable for oral use, topical application, parenteral administration, inhalation, insufflation or as a suppository.

14. A use as claimed in claim 9 wherein the cancer is selected from breast cancer, leukaemia, cervical cancer, ovarian cancer, bowel cancer, lung cancer and colon cancer.

15. A use or pharmaceutical composition as claimed in claim 14 wherein the cancer is an oestrogen receptor negative breast cancer.

16. A use or pharmaceutical composition as claimed in claim 15 wherein the oestrogen receptor negative cancer is selected from MDANQ01, MDA MB-231 and MDA-468.

17. A use or pharmaceutical composition as claimed in claim 14 wherein the cancer is a leukaemia selected from MOLT-4 and K562.

18. A use or pharmaceutical composition as claimed in claimed in claim 14 wherein the glycosaminoglycans are from whelk species.

19. A use or pharmaceutical composition as claimed in claim 14 wherein the glycosaminoglycans are from cockle species.

20. A pharmaceutical composition as claimed in claim 10 comprising one or more further anti-cancer agents selected from an anti-proliferative agent, antineoplastic agent, a cytostatic agent, an anti-invasion agent, and inhibitor of growth factor function, an angiogenic agents, an antisense therapy, a gene therapy, an immunotherapy, and one or more glycosaminoglycans having anti-cancer properties isolated from species other than whelk and/or cockle.

21. A method of inducing apoptosis in cancerous cells, the method comprising the administering of an effective amount of an isolated extract comprising glycosaminoglycans from whelk and/or cockle species.

22. A method as claimed in claim 22 comprising administering an isolated extract comprising glycosaminoglycans from whelk and/or cockle species.

23. An isolated extract, use, pharmaceutical composition, or method as claimed in claim 1 wherein the glycosaminoglycans are extracted from whelks of the family Buccinidae.

24. An isolated extract, pharmaceutical composition, use or method as claimed in claim 23 wherein the whelk is from the genus Buccinum.

25. An isolated extract, use, pharmaceutical composition or method of claim 1 wherein the glycosaminoglycans are extracted from cockles of the family Cardiidae.

26. An isolated extract, use, pharmaceutical composition or method as claimed in claim 25 wherein the cockle is Cerastoderma edule.

27. An isolated heparan or heparan sulphate-like GAG sulphate extract from whelk and/or cockle species.

28. An isolated extract as claimed in claim 27 comprising a lower molecular weight component having a molecular weight in the range of 0.25 KDa to 300 KDa.

29. A heparan sulphate as claimed in claim 27 comprising a higher molecular weight component comprising a molecular weight in the range of between 300 KDa and 1200 KDa.

30. An isolated extract as claimed in claim 27 comprising glycosaminoglycans from a whelk of the family Buccinidae and/or a cockle of the family Cardiidae.

31. A pharmaceutical composition comprising an isolated heparan sulphate or heparan sulphate-like GAG extract of claim 27.

32. A pharmaceutical composition comprising a whelk and/or cockle heparan sulphate or heparan sulphate-like GAG.

33. Use of a pharmaceutical composition comprising heparan sulphate from whelk and/or cockle species for the prevention and/or treatment of cancer and/or cancer metastasis.

34. A pharmaceutical composition comprising at least one isolated glycosaminoglycan for the prevention and/or treatment of leukaemia.

35. The use of a glycosaminoglycan in the prevention and/or treatment of leukaemia.

36. A pharmaceutical composition comprising at least one glycosaminoglycan for use in the prevention and/or treatment of leukaemia.

37. An isolated extract, use, pharmaceutical composition or method, substantially as described herein with reference to the accompanying drawings.
